(19) United States
(12) Patent Application Publication
(10) Pub. No.: US 2021/0290972 A1
(43) Pub. Date: Sep. 23, 2021

(54) BODY ILLUMINATION SYSTEM USING BLUE LIGHT

(71) Applicants: Giovanna WAGENAAR CACCIOLA, EINDHOVEN (NL); Adriaantje P. MOUWS-VAN ROSSUM, EINDHOVEN (NL); Claudia MUTTER, EINDHOVEN (NL); Matthias BORN, EINDHOVEN (NL); Ulrich NIEMANN, EINDHOVEN (NL); Victoria C.P. KOLB-BACHOFEN, EINDHOVEN (NL); Jorg LIEBMANN, EINDHOVEN (NL); Christoph V. SUSCHEK, EINDHOVEN (NL); Christian OPLAENDER, EINDHOVEN (NL)

(72) Inventors: Giovanna WAGENAAR CACCIOLA, EINDHOVEN (NL); Adriaantje P. MOUWS-VAN ROSSUM, EINDHOVEN (NL); Claudia MUTTER, EINDHOVEN (NL); Matthias BORN, EINDHOVEN (NL); Ulrich NIEMANN, EINDHOVEN (NL); Victoria C.P. KOLB-BACHOFEN, EINDHOVEN (NL); Jorg LIEBMANN, EINDHOVEN (NL); Christoph V. SUSCHEK, EINDHOVEN (NL); Christian OPLAENDER, EINDHOVEN (NL)

(73) Assignee: KONINKLIJKE PHILIPS ELECTRONICS N.V., Eindhoven (NL)

(21) Appl. No.: 12/935,959

(22) PCT Filed: Apr. 7, 2009
(86) PCT No.: PCT/IB2009/051453

§ 371 Date: Jul. 19, 2012

(30) Foreign Application Priority Data
Apr. 10, 2008 (EP) 08154291.2

(51) Int. Cl. A61N 5/06

(52) U.S. Cl. CPC A61N 5/0613; A61N 2005/0663; A61N 2005/0642; A61N 2005/0637; A61N 2005/0652

(57) ABSTRACT

The invention relates to a body illumination system comprising one or more light sources, said light sources being configured for emitting light in a wavelength range of 410-470 nm. The system is configured for exposing to said emitted light a surface area of 0.5-2.0 m² of said body such that the power density of said emitted light on said surface area of said body is in the range of 20-120 mW/cm², more preferably 30-100 mW/cm². Alternatively, the system is configured for exposing to said emitted light a surface area of 10-60 cm′ of said body such that the power density of said emitted light on said surface area of said body is in the range of 10-30 mW/cm². The body illumination system is for use in treating physical dysfunction, such as hypertension and erectile dysfunction.

### FIELD OF THE INVENTION

The invention relates to an illumination system. More specifically, the invention relates to an illumination system configured for illuminating a body for treatment of physical dysfunction.

### BACKGROUND OF THE INVENTION

Hypertension (high blood pressure) is a worldwide epidemic. It is predicted that by 2025 there may be more than 1.5 billion sufferers of this disease worldwide. It is reported that 7.1 million deaths, 13% of all mortality, is due to complications from hypertension (stroke, heart and kidney failure). Despite risk awareness, people work longer, exercise less, and don't eat well. This, in combination with an aging population, will enlarge the prevalence of hypertension.

Blood pressure is determined by the amount of blood pumped by the heart and the size and condition of the arteries. Many other factors can affect the blood pressure, such as volume of water in the body, salt content in the body, condition of the kidneys, nervous system, the level of various hormones in the body, and the contraction or relaxation of the blood vessels.

Nitric Oxide (NO) is used by the endothelium of blood vessels to signal the surrounding smooth muscle to relax, thus dilating the artery and increasing blood flow. For instance, NO released from nitro-glycerine dilates the coronary arteries and therefore is applied for diseases, such as angina pectoris or during a heart infarct. Furthermore, NO released by autonomic nerves in the penis causes the local dilation of blood vessels (vasodilation) responsible for penile erection.

US 2006/015156 discloses a method of treating a patient having a condition selected from the group including hypertension, hypertensive crises, angina pectoris, chronic arthritis, erectile dysfunction, cerebral ischemia, and chronic skin ulcers. The disclosed method includes the step of administering to the patient a therapeutically effective amount or dose, or series of amounts or doses, of ultraviolet (UV)light.

Application of UV light is limited due its toxicity and related risk of inducing skin cancer. In addition, at high intensities, UV light causes skin inflammation within a very short period of time. Moreover, UV light may be readily absorbed by the skin and is, therefore, prevented from interacting with the blood.

There exists a need in the art for an improved body illumination system and method for treating physical dysfunction.

### SUMMARY OF THE INVENTION

A body illumination system is proposed that comprises one or more light sources. The light sources are configured for emitting blue light in a wavelength range of 420-470 nm, preferably 430-470 nm. The system is configured for exposing a surface area of 0.5-2.0 m² of the body to the emitted light. The configuration of the system is such that the power density of the emitted light on the surface area of said body is in the range of 20-120 mW/cm², more preferably 30-100 mW/cm². The distance between the surface and the light sources is preferably 10 cm to 1 m, preferably 10 cm to 60 cm.

The applicant has found that such an illumination system can be used effectively for treating physical dysfunction of a patient. It should be appreciated that a patient includes both a human being and an animal. It has been found that blue triggers the production of nitric oxide in blood by breaking the bond between NO and hemoglobin and releasing the free NO radical in the blood and also by stimulating the endothelial cells of the blood vessel wall to produce endogenous NO. In contrast to UV light, blue light can be applied at much higher intensities and doses at a non-toxic level. In addition, blue light penetrates deeper into the skin as compared to UV light. Therefore, blue light may be absorbed much better by the lower lying blood vessels as compared to UV light. The light needs to be absorbed by the superficial blood vessels of the skin in order to enter the systemic circulation and reduce blood pressure in the whole circulatory system. For the blood pressure reduction application, the illumination time is in the order of up to one hour in order to achieve a longer lasting effect.

Furthermore, the use of anti-hypertensive drugs can be reduced. The use of anti-hypertensive drugs is often hampered by heavy side effects such as dizziness, headache, fluid build-up in legs, dry irritating cough, kidney failure, allergic reactions, a decrease in white blood cells and swelling of tissues, sleep disturbances, fatigue, lethargy and erectile dysfunction.

The applicant has also found that the illumination system with the parameter settings as defined in claim 9 can also be used for treating erectile dysfunction. Preferably, the distance between the surface and the light sources is in the range of 10-30 cm for this application.

The drug Viagra causes the autonomic nerves of the penis to produce NO, which results in a local dilation of blood vessels (vasodilatation). This results in a penile erection. In particular, due to the thin skin of the penis and due to the large number of blood vessels, blue light effectively reaches the blood and the endothelial cells, resulting in a strong production of NO. As a result, vasodilatation leads to an increased blood perfusion, causing the penis erection. The illumination time may be very short in order to achieve this temporary effect for a given duration.

The embodiment as defined in claim 2 provides for a highly effective type of light source to accomplish a body illumination system with the required parameters as defined in claim 1. Other light sources, such as high intensity discharge lamps, may be applied, however, these generate more heat to which the body is exposed. Furthermore, such light sources generate light at other visible wavelengths, which is experienced as annoying by users. The high power blue light sources may e.g. be Luxeon® LEDs. The LEDs may have an optical power in the range of 500-600 mW and an electric power in the range of 1.8-2.2 Watt.

The embodiment as defined in claim 3 has the advantage of efficient placement and control of the light sources.

The embodiment as defined in claims 5 and 6 provides for effective cooling of the system using high power light sources.

The embodiment as defined in claim 7 allows the system to be controlled in dependence on the result of a blood pressure test. As an example, the system may be used only if the blood pressure exceeds a particular threshold.

The embodiment as defined in claim 8 allows the system to be remotely controlled, e.g. by a physician. The operating parameters of the body illumination systems may be set remotely. As an example, the result of a blood pressure test may enable the physician to set the appropriate operating parameters. The physician may also send the operating parameters in a different way (e.g. by e-mail or text-messages (e.g. sms)) such that the patient can upload the parameters in the body illumination system.

US 2003/147241 discloses a lighting system suitable for chromotherapy comprising a plurality of light fixtures mounted through walls of a tub basin to project light of different colors into the water in the tub. In the publication, the belief is reported that when a human is exposed to prolonged red light, the person's blood pressure will increase, while with blue light, that person's blood pressure will decrease. However, the specific operating parameters for which this effect is obtained are neither disclosed nor suggested. The applicant has found that the body illumination system with the claimed operating parameters provides for an effective treatment of physical dysfunction while severely reducing the disadvantageous effects occurring with UV treatment.

Hereinafter, embodiments of the invention will be described in further detail. It should be appreciated, however, that these embodiments may not be construed as limiting the scope of protection for the present invention.

### BRIEF DESCRIPTION OF THE DRAWINGS

In the drawings:

FIGS. 1A-1C show schematic illustrations of a body illumination system according to various embodiments of the invention;

FIGS. 2A-2C show a schematic illustration of details of a body illumination system as shown in FIGS. 1A-1C;

FIGS. 3A-3C show in-vitro experimental results of the production of nitric oxide for LED light of different wavelengths; and

FIGS. 4A-4D show in-vitro experimental results of the toxic effects of human fibroblasts in dependence on the light wavelength, for different wavelengths, and the light dose.

### DETAILED DESCRIPTION OF THE DRAWINGS

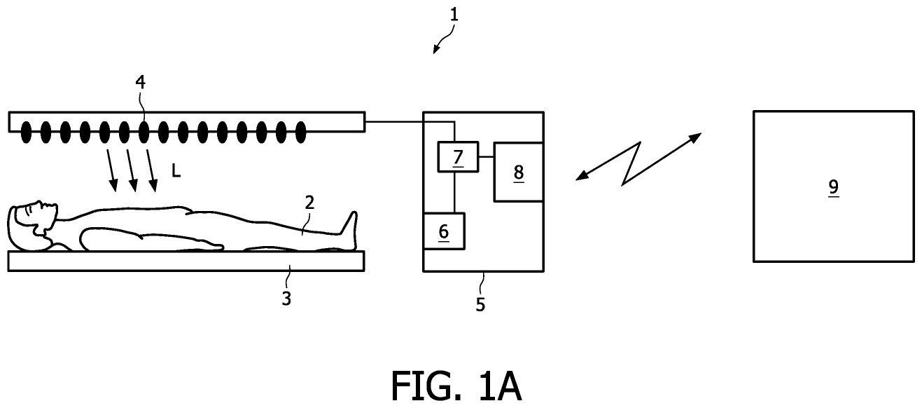

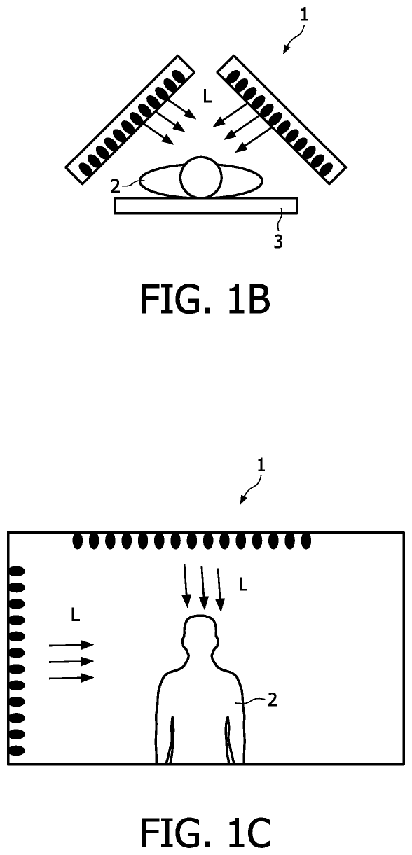

FIGS. 1A-1C provide schematic illustrations of a body illumination system 1. In FIG. 1A, a patient 2 lies on a bed 3 to be illuminated by the body illumination system 1. The body illumination system 1 comprises a plurality of light sources 4 configured for emitting light L on the patient 2 in a sunbed-like arrangement.

In FIG. 1B, two panels, oriented at an angle of 45° with respect to the bed 3, are provided that comprise light sources 4.

It should be appreciated that the light sources 4 are not necessarily provided over the entire body of the patient 2.

It should also be appreciated that the patient 2 is not necessarily on a bed. The body illumination system 1 may e.g. be comprised in a room R, the light sources 4 being accommodated in the walls of the room R, or being integrated in a standing system, as shown in FIG. 1C.

The body illumination system 1 comprises a control unit 5 containing a blood pressure sensor 6, a controller 7 and a data transceiver 8. It is noted that the control unit 5 may also be provided for the body illumination systems 1 of FIGS. 1B and 1C. The control unit 5 may comprise further modules, including e.g. a memory module for data storage.

The control unit 5 is configured for controlling the light sources 4 by means of the controller 7. The blood pressure sensor 6 is configured for measuring the blood pressure of the patient 2 when in contact with the body. Of course, a separate blood pressure sensor may also be used and the blood pressure may then be input into the body illumination system 1. The controller 7 may be triggered to activate the light sources (and, possibly, to set operating parameters of the light sources) by the result of the measurement of the blood pressure by the blood pressure sensor 6. Data collected by the control unit 5 may be transmitted to a remote external system 9 via the data transceiver 8. Also, the data transceiver 8 of the control unit 5 may receive data and/or control instructions from the remote system 9 for operating the body illumination system 1. Data transfer may be performed via a wired and/or a wireless network. The controller 7 may include a timer for programming or determining the operative time of the body illumination system 1.

The light sources 4 are preferably high power light-emitting diodes (LEDs). These LEDs 4 may be Luxeon® LEDs or Lumileds. The LEDs have an optical power in the range of 500-6500 mW and an electric power of 1.8-2.2 W. The number of LEDs may vary between 10 and 1,000, dependent on the application.

The drivers of the LEDs 4 may be provided in a separate unit (see e.g. FIG. 1A) or can be placed on the back side the LEDs.

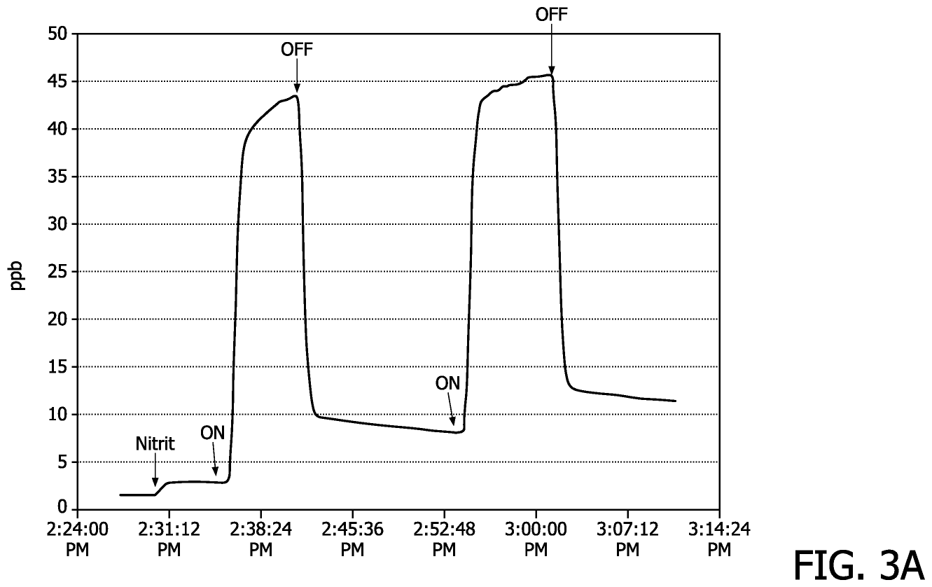

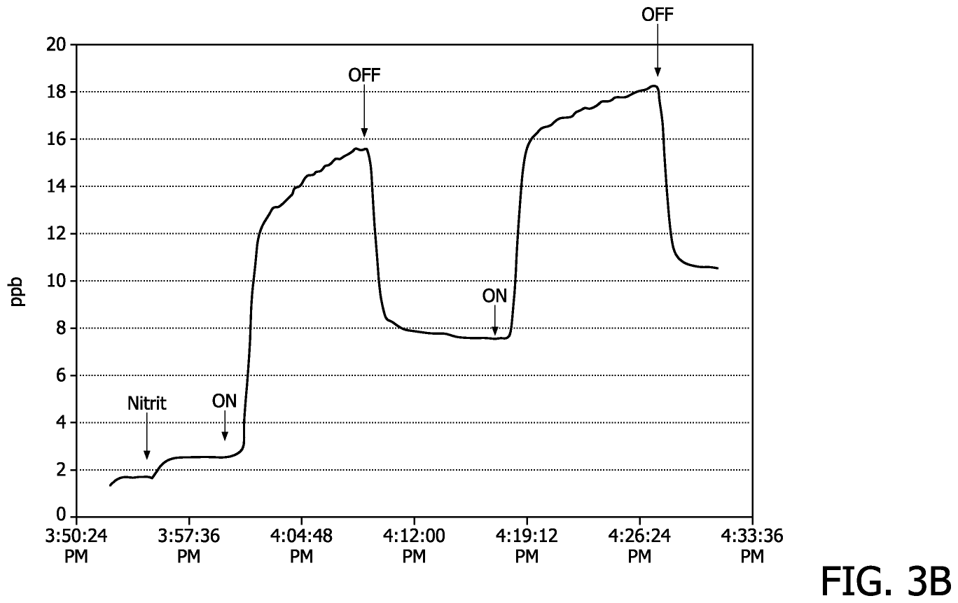

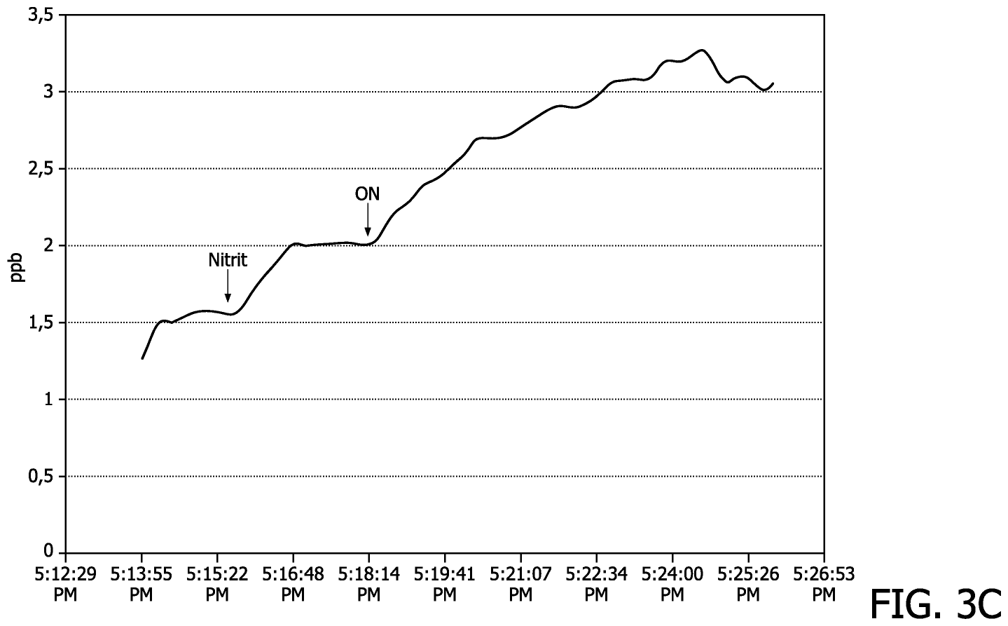

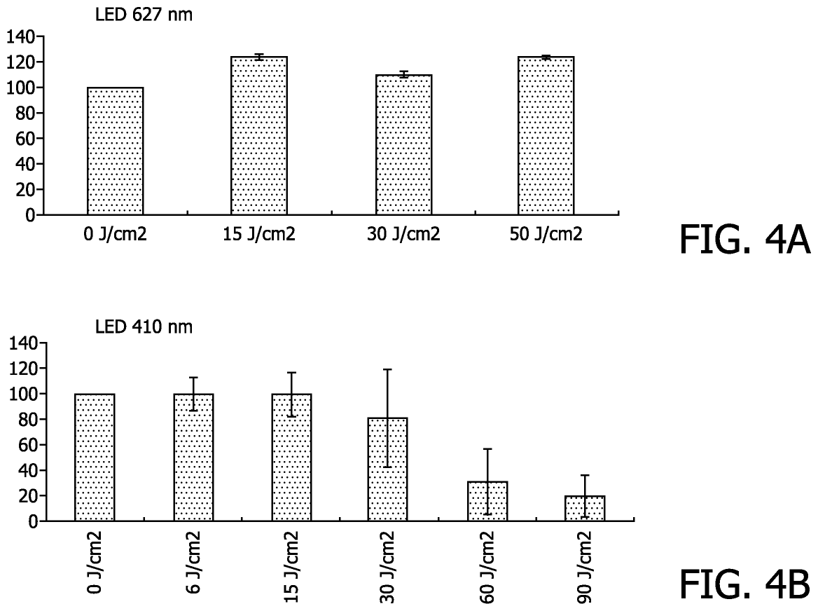

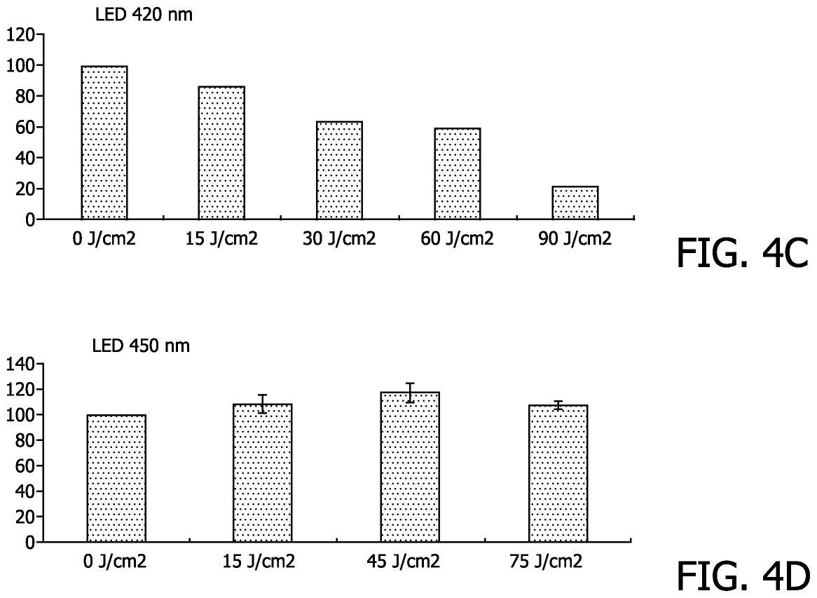

For the treatment of physical dysfunction, in particular hypertension and erectile dysfunction, the light sources 4 emit blue light, thereby simultaneously reducing disadvantageous effects resulting from the use of UV light, as will be described in further detail with reference to FIGS. 3A-3C and 4A-4D. The wavelength of the emitted light L is in the range of 410-500 nm. In particular, a significant reduction of toxic effects may be obtained for wavelengths in the range of 425-470, 440-470 nm or even in the range of 450-465 nm.

The efficiency of the therapeutic treatment requires that a considerable surface area of the body is illuminated by the body illumination system 1. To that end, the body illumination system 1 should be configured such that it is capable of illuminating a surface area of 0.5-2.0 m² for the treatment of hypertension. For erectile dysfunction, a surface area of 10-60 cm² is sufficient. The efficiency of the therapeutic treatment is determined by the power density of the light L. For the treatment of hypertension, the power density of the emitted light L of the body illumination system 1 is in the range of 20-120 mW/cm², 40-100 mW/cm² or 50-80 mW/cm². For the treatment of erectile dysfunction a power density in the same range may be applied. The lower limit of the power density is determined by the effectiveness of the treatment, while the upper limit is determined by the fact that the heat and light become unpleasant for a patient. A typical treatment time varies between 1 minute and 1 hour, such as 15 minutes.

Depending on the application, the distance between the LEDs 4 and the surface S of the patient 2 is in the range of 10 cm to 150 cm. For the treatment of hypertension, the distance is preferably in the range of 10 cm to 100 cm, in the range of 20 cm to 60 cm and even in the range of 25 cm to 55 cm, such as 40 cm. Treatment of erectile dysfunction may be obtained using smaller distances, e.g. in the range of 10-50 cm or 15-30 cm, such as 20 cm.

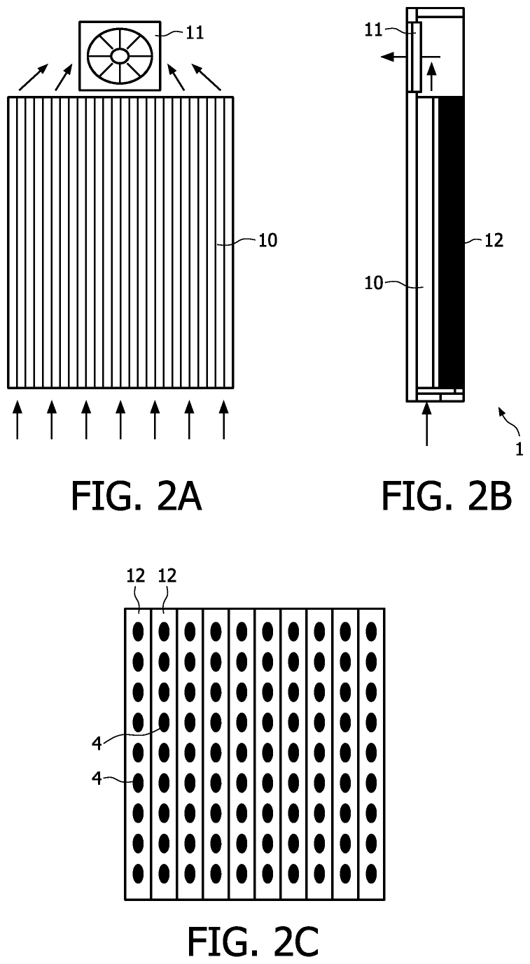

FIGS. 2A-2C show a schematic illustration of details of a body illumination system 1. FIG. 2A shows the body illumination system 1 comprising a channeled heat sink plate 10 in combination with a fan 11. The arrows indicate the air flow for cooling. FIG. 2B illustrates a cross-section of the body illumination system 1, whereas FIG. 2C illustrates a front view containing the LEDs 4. The LEDs 4 are provided in modules 12. LED drivers can be provided on the back side of these modules 12.

In particular, the LEDs 4 are mounted on printed circuit boards and are equipped with the heat sink plate 10. The heat sink plate comprises metal. The fan 11 is mounted on top of the heat sink plate 10 to provide effective heat removal by ventilation. FIGS. 3A-3C and FIGS. 4A-4D show in-vitro experimental results of blue light illumination of endothelial cells in nitride solutions with a pH of 5.5. These conditions are believed to correspond to the conditions existing in human skin.

In FIGS. 3A-3C, the horizontal axis is a time axis.

FIGS. 3A and 3B show a marked increase of nitric oxide (NO) production (vertical axis) immediately after switching on the light illumination system 1 using LEDs of 500-600 mW optical power and 1.8-2.2 W electric power at wavelengths of 410 nm and 420 nm, respectively. When the illumination system 1 is switched off, the NO production drops.

FIG. 3C illustrates the NO production for an illumination experiment with LEDs 4 at a wavelength of 450 nm. Again, the NO production is found to increase upon switching on the body illumination system 1, although less markedly than for wavelengths of 410 nm and 420 nm. The operating parameters of the body illumination system 1 were chosen such that no significant warming of the cells occurred.

In summary, the applicant has found that blue light, i.e. light outside the UV wavelength region, is capable of producing non-enzymatic nitric oxide from nitric solutions with a pH of 5.5.

FIGS. 4A-4D illustrate measurements as to the toxic effects of the blue light illumination for various doses of light (in J/cm²) along the horizontal axis. The vertical axis has arbitrary units, the value of which is set to 100 for a dose of 0 J/cm², at other doses the measured value is relative to the value at 0 J/cm². The toxic effect was measured by a cytotoxicity measurement. Treatment was considered not toxic if practically no or no died cells were counted during a cell count of living cells.

FIG. 4A illustrates reference measurements on a human skin fibroblast sample treated with light of 627 nm wavelength. Toxic effects were not observed up to a dose of 50 J/cm².

FIGS. 4B and 4C illustrate measurements on a human skin fibroblast sample treated with light of 410 nm and 420 nm wavelength. Toxic effects were observed to depend on the illumination dose to which the sample is exposed.

FIG. 4D illustrates measurements on a human skin fibroblast sample treated with light of 450 nm wavelength. Surprisingly, no toxic effects were observed over the complete radiation dose range of 0-75 J/cm².

Finally, it is noted that the patient may administer supplements in order to increase the efficiency of the light treatment. The use of supplements such as antioxidants in combination with light treatment is recommended, because of the protective effect on the NO radical and the maintenance of stability in the endothelial cell membranes. The antioxidants protect cells against the effects of free radicals produced during normal oxygen metabolism and reduce therefore the peroxidation reaction and the loss of free nitric oxide, the neurotransmitter, which relaxes the walls of the blood vessels. Antioxidant supplements that may be used include L-taurine and Selenium. L-taurine is an amino acid, found in eggs, dairy products, meat and fish proteins and in nutraceutical form. Selenium ensures that the endothelial cells will be free to produce maximal nitric oxide, resulting in improved cardiovascular health.

1. A body illumination system comprising one or more light sources, said light sources being configured for emitting light in a wavelength range of 410-470 nm, preferably 430-470 nm, wherein said system is configured for exposing to said emitted light a surface area of 0.5-2.0 m² of said body such that the power density of said emitted light on said surface area of said body is in the range of 20-120 mW/cm², more preferably 30-100 mW/cm².

2. The body illumination system according to claim 1, wherein said light sources are high power light-emitting diodes (LEDs).

3. The body illumination system according to claim 2, wherein said LEDs are arranged in modules, each module comprising a plurality of said LEDs.

4. The body illumination system according to claim 1, wherein said light sources have an optical power of 400-700 mW.

5. The body illumination system according to claim 1, further comprising a heat sink configured for transporting heat away from said light sources.

6. The body illumination system according to claim 1, further comprising a fan.

7. The body illumination system according to claim 1, further comprising a blood pressure sensor.

8. The body illumination system according to claim 1, further comprising at least one of a data transmitter and a data receiver configured for transmitting data from said body illumination system and receiving data at said body illumination system, respectively.

9. A body illumination system comprising one or more light sources, said light sources being configured for emitting light in a wavelength range of 420-470 nm, preferably 430-470 nm, wherein said system is configured for exposing to said emitted light a surface area of 10-60 cm² of said body, such that the power density of said emitted light on said surface area of said body is in the range of 10-30 mW/cm².

10. A method of treating a patient suffering from physical dysfunction comprising the step of exposing said person to light in a wavelength range of 410-470 nm, preferably 440-470 nm, to receive a therapeutically effective dose of said light.

11. The method according to claim 10, wherein said physical dysfunction is selected from hypertension and erectile dysfunction.

12. The method according to claim 10, further comprising the step of exposing said person to a system comprising a plurality of light sources for emitting said light, such that a surface area of 0.5-2.0 m² of said person is exposed, wherein the power density of said emitted light on said surface area is in the range of 20-120 mW/cm²; more preferably 30-100 mW/cm².

13. The method according to claim 10, further comprising the step of exposing the penis of said person to a system comprising a plurality of light sources for emitting said light, such that a surface area of 10-60 cm² of said penis is exposed, wherein the power density of said emitted light on said surface area is in the range of 10-30 mW/cm².
